(19) United States
(12) Patent Application Publication
(10) Pub. No.: US 2008/0207544 A1
(43) Pub. Date: Aug. 28, 2008

(54) Compositions And Methods For Delivery Of Agents Into Cells

(76) Inventor: Sean M. Sullivan, San Diego, CA (US)

Correspondence Address: Beusse Wolter Sanks Mora & Maire, 390 N. ORANGE AVENUE, SUITE 2500, ORLANDO, FL, 32801, US

(21) Appl. No.: 11/814,180

(22) PCT Filed: Jan. 25, 2006
(86) PCT No.: PCT/US06/02881

§ 371 Date: Jul. 18, 2007

Related U.S. Application Data
(60) Provisional application No. 60/646,901, filed on Jan. 25, 2005.

(51) Int. Cl. A61K 31/7115; A61K 31/683

(52) U.S. Cl. 514/44; 514/77

(57) ABSTRACT

Disclosed herein are novel compositions for delivery of biologically active agents into cells comprising a cationic amphiphile and a complementary lipid composition. Also disclosed are methods of making and using such novel compositions.

This application claims the benefit of U.S. provisional Application Ser. No. 60/646,901 filed Jan. 25, 2005.

### BACKGROUND

It is widely hoped that gene therapy will provide a long lasting and predictable form of therapy for certain disease states, and it is likely the only form of therapy suitable for many inherited diseases. There remains however a critical need to develop compounds that facilitate entry of functional genes into cells, and whose activity in this regard is sufficient to provide for in vivo delivery of genes or other such biologically active therapeutic molecules in concentrations thereof that are sufficient for intracellular therapeutic effect.

In as much as compounds designed to facilitate intracellular delivery of biologically active molecules must interact with both non-polar and polar environments (in or on, for example, the plasma membrane, tissue fluids, compartments within the cell, and the biologically active molecule itself), such compounds are designed typically to contain both polar and non-polar domains. Compounds having both such domains may be termed amphiphiles, and many lipids and synthetic lipids that have been disclosed for use in facilitating such intracellular delivery (whether for in vitro or in vivo application) meet this definition. One particularly important class of such amphiphiles is the cationic amphiphiles. In general, cationic amphiphiles have polar groups that are capable of being positively charged at or around physiological pH, and this property is understood in the art to be important in defining how the amphiphiles interact with the many types of biologically active (therapeutic) molecules including, for example, negatively charged nucleic acid sequences such as DNA. Though some strides have been made in formulating compositions that will assist in the intracellular delivery of biologically active compounds into cells, there remains a need for easy to obtain compositions with enhanced transfection efficiency.

### SUMMARY

One aspect of the invention relates to novel cationic amphiphile and helper transfection reagent compositions, and there use to form cationic amphiphile/biologically active agent complexes for the delivery of molecules, polymers, nucleic acids and genes to cells.

Another aspect of the invention pertains to a process for obtaining an expression product by delivering a polynucleotide to a cell, comprising the step of associating a first and second lipid, and a polynucleotide to form a complex. Then, delivering the complex to the cell and expressing the polynucleotide in the cell.

Another aspect of the invention pertains to a process for obtaining an expression product in vivo, comprising forming a complex with a first lipid, a second lipid and a polynucleotide. Then, delivering the complex to a cell in a mammal which expresses the polynucleotide. These and other advantageous aspects are described further herein.

### BRIEF DESCRIPTION OF THE DRAWINGS

FIG. 1 shows the chemical structures of a complimentary lipid composition whose tradename is Lym-X-Sorb™.

FIG. 2 illustrates the ability of Lym-X-Sorb™ to rearrange into an inverse hexagonal phase.

FIGS. 3-6 represent photographs of cells showing the successful transfection of cells with complexes according to different embodiments of the subject invention.

FIGS. 7-9 show graphs illustrating the ability of complex embodiments to transfect different cell types.

FIG. 10. Upregulation of CXCR4-expression on HUVEC in response to recombinant rat VEGF164. HUVEC are incubated with 100 ng/ml rrVEGF164 for different time periods after which surface-expression of CXCR4 is determined by Flow Cytometry.

FIG. 11. Total cell-associated fluorescence of HUVEC after incubation with CXCR4-targeted- and control-liposomes. After attachment of HUVEC to the plate media was replaced with media with or without 100 ng/ml rrVEGF164. After 16 hrs the cells were incubated for 1 hour with fluorescently labeled liposomes.

FIG. 12. In vitro gene transfer activity of lipoplexes modified with different lipids. Complexes with DOPE-MPB-Cys, DOPE or CXCR4-lipopeptide were incubated with RG2 cells (1 μg pLC0888/well). Each point is an average of three wells ±SD.

FIG. 13. Transfection of B16 cells by implementation of FGF receptor targeted lipoplexes.

### DETAILED DESCRIPTION

### Definitions

The term “lipid” is used in its conventional sense as a generic term encompassing fats, lipids, the alcohol-ether-soluble constituents of protoplasm, which are insoluble in water. Lipids compose the fats, fatty oils, essential oils, waxes, steroids, sterols, phospholipids, glycolipids, sulpholipids, aminolipids, chromolipids (lipochromes), and fatty acids. The term encompasses both naturally occurring and synthetically produced lipids. Preferred lipids in connection with the present invention are: phospholipids, including phophatidylcholines and phosphatidylethanolamines, and sphingomyelins. Where there are fatty acids, they could be 12-24 carbons in length, containing up to 6 unsaturations (double bonds), and linked to the backbone by either acyl or ether linkages. Where there is more than one fatty acid linked to the backbone, the fatty acids could be different (asymmetric), or there may be only 1 fatty acid chain present, e.g. lysolecithins. Mixed formulations are also possible, particularly when the non-cationic lipids are derived from natural sources, such as lecithins (phosphatidylcholines) purified from egg yolk, bovine heart, brain, or liver, or soybean. Steroids and sterols, particularly cholesterol, and sterols substituted at the 3b position.

The term “cationic lipid” is used herein to encompass any lipid of the invention (as defined above) which is cationic. The lipid will be determined as being cationic when the lipid has a positive charge (at physiological pH) as measurable by instrumentation utilized at the time of the measurement. Where there are fatty acids present on the cationic lipid, they could be 12-24 carbons in length, containing up to 6 unsaturations (double bonds), and linked to the backbone by either acyl or ether linkages; there could also only be one fatty acid chain linked to the backbone. Where there is more than one fatty acid linked to the backbone, the fatty acids could be different (asymmetric). Mixed formulations are also possible. A cationic lipid serve as an amphiphile, as defined herein below.

The term “liposome” encompasses any compartment enclosed by a lipid bilayer. Liposomes are also referred to as lipid vesicles. In order to form a liposome the lipid molecules comprise elongated non-polar (hydrophobic) portions and polar (hydrophilic) portions. The hydrophobic and hydrophilic portions of the molecule are preferably positioned at two ends of an elongated molecular structure. When such lipids are dispersed in water they spontaneously form bilayer membranes referred to as lamellae. The lamellae are composed of two mono layer sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell. Thus, the bilayer of a liposome has similarities to a cell membrane without the protein components present in a cell membrane. As used in connection with the present invention, the term liposome includes multilamellar liposomes, which generally have a diameter in the range of 1 to 10 micrometers and are comprised of anywhere from two to hundreds of concentric lipid bilayers alternating with layers of an aqueous phase, and also includes unilamellar vesicles which are comprised of a single lipid layer and generally have a diameter of 20 to 100 nanometers which vesicles can be produced by subjecting multilamellar liposomes to ultrasound.

The term “cationic liposome” as used herein is intended to encompass any liposome as defined above which is cationic. The liposome is determined as being cationic when present in physiological pH. It should be noted that the liposome itself is the entity which is being determined as cationic meaning that the liposome which has a measurable positive charge within its physiological pH may, within an in vivo environment, become attached to other substances. Those other substances may be negatively charged and thereby result in the formation of a structure which does not have a positive charge. The charge and/or structure of a liposome of the invention present within an in vivo environment has not been precisely determined. However, in accordance with the invention a cationic liposome of the invention will be produced using at least some lipids which are themselves cationic. The liposome need not be comprised completely of cationic lipids but must be comprised of a sufficient amount of cationic lipid such that when the liposome is formed and placed within an in vivo environment at physiological pH the liposome initially has a positive charge.

The term “biologically active agent/cationic amphiphile complex” refers to a combination of a biologically active agent, e.g., polynucleotide which may be an RNA or a DNA sequence, which is combined with at least one cationic amphiphile such as one or more cationic lipids as defined above and may include neutral lipids. When DNA sequences and cationic lipids are combined, they will spontaneously form complexes which are not classical liposomes. Complexes may form through noncovalent interactions such as electrostatic interactions, hydrogen bonding interactions, and hydrophobic interactions.

The terms “admix” or “admixing” refers to a process of combining two or more compounds together such that contact and allow any covalent or non-covalent interaction to occur.

The “biologically active agent” as used herein refers to a agent having the potential to react with biological components. More particularly, biologically active agents utilized in this specification are designed to change the natural processes associated with a living cell. For purposes of this specification, a cellular natural process is a process that is associated with a cell before delivery of a biologically active agent. In this specification, the cellular production of, or inhibition of a material, such as a protein, caused by a human assisting a molecule to an in vivo cell is an example of a delivered biologically active agent. Pharmaceuticals, proteins, peptides, polypeptides, enzyme inhibitors, hormones, cytokines, antigens, viruses, oligonucleotides, enzymes and polynucleotides are examples of biologically active agents.

The terms “peptide and polypeptide” refer to a series of amino acid residues, more than two, connected to one another by amide bonds between the beta or alpha-amino group and carboxyl group of contiguous amino acid residues. The amino acids may be naturally occurring or synthetic. Polypeptide includes proteins and peptides, modified proteins and peptides, and non-natural proteins and peptides. Enzymes are proteins evolved by the cells of living organisms for the specific function of catalyzing chemical reactions. A chemical reaction is defined as the formation or cleavage of covalent or ionic bonds. Bioactive agents may be used interchangeably with biologically active agent for purposes of this application.

“Deliver” or “delivered” means that the biologically active agent becomes associated with the cell or organism, including introduced into the cell. The agent can be in the circulatory system, intravessel, extracellular, on the membrane of the cell or inside the cytoplasm, nucleus, or other organelle of the cell.

Parenteral routes of administration include intravascular (intravenous, intraarterial), intramuscular, intraparenchymal, intradermal, subdermal, subcutaneous, intratumor, intraperitoneal, intrathecal, subdural, epidual, intraocular, and intralymphatic injections that use a syringe and a needle or catheter. An intravascular route of administration enables a polymer or polynucleotide to be delivered to cells more evenly distributed and more efficiently expressed than direct injections. Intravascular herein means within a tubular structure called a vessel that is connected to a tissue or organ within the body. Within the cavity of the tubular structure, a bodily fluid flows to or from the body part. Examples of bodily fluid include blood, cerebrospinal fluid (CSF), lymphatic fluid, or bile. Examples of vessels include arteries, arterioles, capillaries, venules, sinusoids, veins, lymphatics, and bile ducts. The intravascular route includes delivery through the blood vessels such as an artery or a vein. An administration route involving the mucosal membranes is meant to include nasal, bronchial, inhalation into the lungs, or via the eyes. Other routes of administration include intraparenchymal into tissues such as muscle (intramuscular), liver, brain, and kidney. Transdermal routes of administration have been effected by patches and ionotophoresis. Other epithelial routes include oral, nasal, respiratory, and vaginal routes of administration. Intraocular administration may involve, for example, injection into the vitreous humor of the eye or by application of drops to the exterior surface of the eye. Such parenteral routes of administration are the typical mode of administration.

The term “polynucleotide” is a term of art that refers to a polymer containing at least two nucleotides. “Nucleotides” contain a sugar deoxyribose (DNA) or ribose (RNA), a base, and a phosphate group. Nucleotides are linked together through the phosphate groups. “Bases” include purines and pyrimidines, which further include natural agents adenine, thymine, guanine, cytosine, uracil, inosine, and natural analogs, and synthetic derivatives of punines and pyrimidines, which include, but are not limited to, modifications which place new reactive groups such as, but not limited to, amines, alcohols, thiols, carboxylates, and alkylhalides. Nucleotides are the monomeric units of nucleic acid polymers. An “oligonucleotide” as used herein is polynucleotide that contains less than 80 monomeric units; oligonucleotides contain from 2 to 80 nucleotides. The term nuclei acid includes deoxyribonucleic acid (DNA) and ribonucleic acid (RNA). The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

DNA may be in the form of anti-sense, plasmid DNA, parts of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, ribozymes, chimeric sequences, or derivatives of these groups. siRNA (small inhibitory RNA), miRNA (micro RNA).

“Anti-sense” is a polynucleotide that interferes with the function of DNA and/or RNA. This may result in suppression of expression. Natural nucleic acids have a phosphate backbone, artificial nucleic acids may contain other type of backbones and bases. These include PNAs (peptide nucleic acids), phosphothionates, and other variants of the phosphate backbone of native nucleic acids. In addition, DNA and RNA may be single, double, triple, or quadruple stranded.

The term “recombinant DNA molecule” as used herein refers to a DNA molecule that is comprised of segments of DNA joined together by means of molecular biological techniques. “Expression cassette” refers to a natural or recombinantly produced polynucleotide molecule that is capable of expressing protein(s). A DNA expression cassette typically includes a promoter (allowing transcription initiation), and a sequence encoding one or more proteins. Optionally, the expression cassette may include trancriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences.

A polynucleotide can be used to modify the genomic or extrachromosomal DNA sequences. This can be achieved by delivering a polynucleotide that is expressed. Alternatively, the polynucleotide can effect a change in the DNA or RNA sequence of the target cell. This can be achieved by homologous recombination, gene conversion, or other, yet to be described, mechanisms.

The term “gene” refers to a nucleic acid (e.g., DNA) sequence that comprises coding sequences necessary for the production of a polypeptide or precursor (e.g., —myosin heavy chain). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends far a distance of about 1 kb or more on either end such that the gene corresponds to the length of the full-length mRNA. The sequences that are located 5′ of the coding region and which are present on the mRNA are referred to as 5′ nontranslated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence which encodes a gene product. The coding region may be present in either a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript

Alternatively, the coding region utilized in the expression vectors of the present invention may contain endogenous enhancers/promoters, splice junctions, intervening sequences, polyadenylation signals, etc. or a combination of both endogenous and exogenous control elements.

The term “isolated” when used in relation to a polynucleotide, as in “an isolated oligonucleotide” or “isolated polynucleotide” refers to a polynucleotide that is identified and separated from at least one contaminant nucleic acid with which it is ordinarily associated in its natural source. Isolated nucleic acid is such present in a form or setting that is different from that in which it is found in nature. In contrast, nonisolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated polynucleotide encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated oligonucleotide, or polynucleotide may be present in single stranded or double-stranded form. When an isolated oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down regulation” or “repression” refers to regulation that decreases production. Molecules (e.g., transcription factors) that are involved in up-regulation or down-regulation are often called “activators” and “repressors,” respectively.

The process of delivering a polynucleotide to a cell has been commonly termed “transfection” or the process of “transfecting” and also it has been termed “transformation”. The polynucleotide could be used to produce a change in a cell that can be therapeutic. The delivery of polynucleotides or genetic material for therapeutic and research purposes is commonly called “gene therapy”. The delivery of nucleic acid can lead to modification of the DNA sequence of the target cell.

The term “transfection” as used herein refers to the introduction of foreign DNA into eukaryotic cells. The term “stable transfection” or “stably transfected” refers to the introduction and integration of foreign DNA that is preserved in progeny of the transfected cell, e.g. introduction and integration into the genome of the transfected cell. The term “stable transfectant” refers to a cell which has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA. The term “naked polynucleotides” indicates that the polynucleotides are not associated with a transfection reagent or other delivery vehicle that is required for the polynucleotide to be delivered to a cell.

A “transfection reagent” or “delivery vehicle” is a agent or agents that bind(s) to or complex(es) with oligonucleotides, polynucleotides, or other desired agents and mediates their entry into cells. Examples of transfection reagents include, but are not limited to, cationic liposomes and lipids, polyamines, calcium phosphate precipitates, histone proteins, polyethylenimine, and polylysine complexes (polyethylenimine and polylysine are both toxic). Typically, when used for the delivery of nucleic acids, the transfection reagent has a net positive charge that binds to the polynucleotide's negative charge. For example, cationic liposomes or polylysine complexes have net positive charges that enable them to bind to DNA or RNA. In a preferred embodiment, the transfection reagent is a cationic amplliphile. Even more preferably, the transfection reagent is SDC and/or Lym-X-Sorb.

Enzyme is a protein that acts as a catalyst. That is a protein that increases the rate of a chemical reaction without itself undergoing any permanent chemical change. The chemical reactions that are catalyzed by an enzyme are termed enzymatic reactions and chemical reactions that are not are termed nonenzymniatic reactions.

“Cell targeting signal” (or abbreviated as the Signal) is intended to mean a molecule that modifies a biologically active agents such as drug or nucleic acid and can direct it to a cell location (such as tissue) or location in a cell (such as the nucleus) either in culture or in a whole organism. By modifying the cellular or tissue location of the foreign gene, the function of the biologically active agent can be enhanced.

The cell targeting signal can be a protein, peptide, lipid, steroid, sugar, carbohydrate, (non-expressing) polynucleic acid or synthetic compound. The cell targeting signal enhances cellular binding to receptors, cytoplasmic transport to the nucleus and nuclear entry or release from endosomes or other intracellular vesicles.

Nuclear localizing signals enhance the targeting of the pharmaceutical into proximity of the nucleus and/or its entry into the nucleus. Such nuclear transport signals can be a protein or a peptide such as the SV40 large T ag NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. These include a short NLS (H-CGYGPKKKRKVGG-OH) or long NLS's (H-CKKKSSSDDEATADSQHSTPPKKKRKVEDPKDFPSELLS-OH and H-CKKKWDDEATADSQHSTPPI(KKRKVEDPKDFPSELLS-OH). Other NLS peptides have been derived from M9 protein (CYNDFGNYNNQSSNFGPMKQGNFGGRSSGPY), E1A (H-CKRGPKRPRP-OH), nucleoplasmin (H-CKKAVKRPAATKKAGQAKKKKL-OH), and c-myc (H-CKKKGPAAKRVKLD-OH).

Signals that enhance release from intracellular compartments (releasing signals) can cause DNA release from intracellular compartments such as endosomes (early and late), lysosomes, phagosomes, vesicle, endoplasrmic reticulum, golgi apparatus, trans golgi network (TGN), and sarcoplasmic reticulum. Release includes movement out of an intracellular compartment into cytoplasm or into an organelle such as the nucleus. Releasing signals include chemicals such as chloroquine, bafilomycin or Brefeldin A1 and the ER-retaining signal (KDEL sequence), viral components such as influenza virus hemagglutinin subunit HA-2 peptides and other types of amphipathic peptides.

Cellular receptor signals are any signal that enhances the association of the biologically active agent with a cell. This can be accomplished by either increasing the binding of the agent to the cell surface and/or its association with an intracellular compartment, for example: ligands that enhance endocytosis by enhancing binding the cell surface. This includes agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl agents, and amphotericin derivatives. In addition viral proteins could be used to bind cells.

Cell targeting signals may be linked to or combined with biologically active agents described above.

Reporter or marker molecules are compounds that can be easily detected. Typically they are fluorescent compounds such as fluorescein, rhodamine, Texas red, cy 5, cy 3 or dansyl compounds. They can be molecules that can be detected by infrared, ultraviolet or visible spectroscopy or by antibody interactions or by electron spin resonance. Biotin is another reporter molecule that can be detected by labeled avidin. Biotin could also be used to attach targeting groups. Reporter or marker molecules may linked to or combined with biologically active agents described above.

Linkage(s) are an attachment that provides a covalent bond or spacer between two other groups (chemical moieties). The linkage may be electronically neutral, or may bear a positive or negative charge. The chemical moieties can be hydrophilic or hydrophobic. Preferred spacer groups include, but are not limited to C1-C12 alkyl, C1-C12 alkenyl, C1-C12 alkynyl, C6-C18 aralkyl, C6-C18 aralkenyl, C6-C18 aralynyl, ester, ether, ketone, alcohol, polyol, amide, amine, polyglycol, polyether, polyamine, thiol, thio ether, thioester, phosphorous containing, and heterocyclic.

Amphipathic, amphiphile or amphiphilic, is used in its conventional sense and pertains to compounds that have both hydrophilic (water-soluble) and hydrophobic (water-insoluble) domains. Hydrophilic groups indicate in qualitative terms that the chemical moiety is water-preferring. Typically, such chemical groups are water soluble, and are hydrogen bond donors or acceptors with water. Examples of hydrophilic groups include compounds with the following chemical moieties; carbohydrates, polyoxyethylene, peptides, oligonucleotides and groups containing amines, amides, alkoxy amides, carboxylic acids, sulfurs, or hydroxyls. Hydrophobic groups indicate in qualitative terms that the chemical moiety is water-avoiding. Typically, such chemical groups are not water soluble, and tend not to form hydrogen bonds. Hydrocarbons are hydrophobic groups.

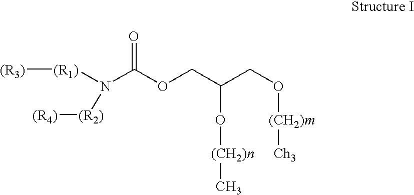

R³ is H, or saturated or unsaturated aliphatic group; R¹ is an alkylamine or polyalkylamine; R⁴ is H, or a saturated or unsaturated aliphatic group; R² is an alkylamine or polyalkylamine; and coefficient (x) is independently 0, 1 or 2 and coefficients (m) and (n) each, independently, ranges from 7 to 29, and the alkyl chains that result therefrom may contain one or more points of unsaturation. In a preferred embodiment, the structure represented by R³—R¹—N—R²—R⁴ in Structure I is spermine or spermidine. In an even more preferred embodiment, the compound represented by Structure I is 1-(N⁴-spermine)-2,3-dilaurylglycerol carbamate; 1-(N⁴-spermidine)-2,3-dilaurylglycerol carbainate; 1-(N⁴-spermine)-2,3-distearylglycerol carbamate; and 1-(N⁴-spermidine)-2,3-distearylglycerol carbamate.

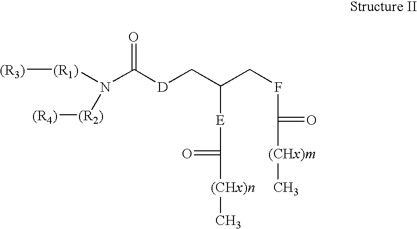

R³ is H, or saturated or unsaturated aliphatic group;
R¹ is an alkylamine or polyalkylamine;
R⁴ is H, or a saturated or unsaturated aliphatic group;
R² is an alkylamine or polyalkylamine;
D, E, and/or F are, independently, O, N, or S; and
coefficient (x) is 2 and coefficients (m) and (n) each, independently, ranges from 7 to 29, and the alkyl chains that result may contain one or more points of unsaturation. For example, the different types of saturated (hexyl(6), heptyl(7), octyl(8), nonyl(9), decyl(10), undecyl(1), dodecyl(12), myristyl(14), palmityl(16), stearyl(18), etc., allyl chains may be monounsaturated, diunsaturated, triunsaturated or tetraunsaturated. In a preferred embodiment, the structure represented by R³—R¹—N—R²—R⁴ in Structure II is spermine or spermidine. In an even more preferred embodiment, the compound represented by Structure II is 1-(N⁴-Spermidine)-2,3-dilauroylglycerol carbamate; and 1-(N⁴-Spermidine)-2,3-distearoylglycerol carbamate.

### Complementary Lipid Composition

In a typical embodiment, the cationic amphiphiles described in Structures I and II above, are combined with a complementary lipid composition (CLC). Such CLC typically comprises a non-esterified fatty acid having 14-18 carbon atoms; a monoglyceride which is a monoester of glycerol and a fatty acid having 14-18 carbon atoms; and a lysophosphatidyl choline in which the fatty acid moiety has 14-18 carbon atoms; that are preferably admixed together. See U.S. Pat. No. 5,972,911 which is incorporated by reference. The different components of the CLC are preferably present as follows: the mole ratio of said monoglyceride to fatty acid is from about 2:1 to about 1:2 and the mole ratio of lysophosphatidyl choline to the sum of monoglycerides and fatty acid is from about 1:3 to about 1:12. A helper reagent maybe added to the cationic amphiphile/CLC combination, such as, but not limited to α-tocopherol, to prevent oxidation of the double bonds in the acyl chains.

### EXAMPLE 1

### Preparation of 1-(N⁴-spermine)-2,3-dilaurylglycerol carbamate (SDC)

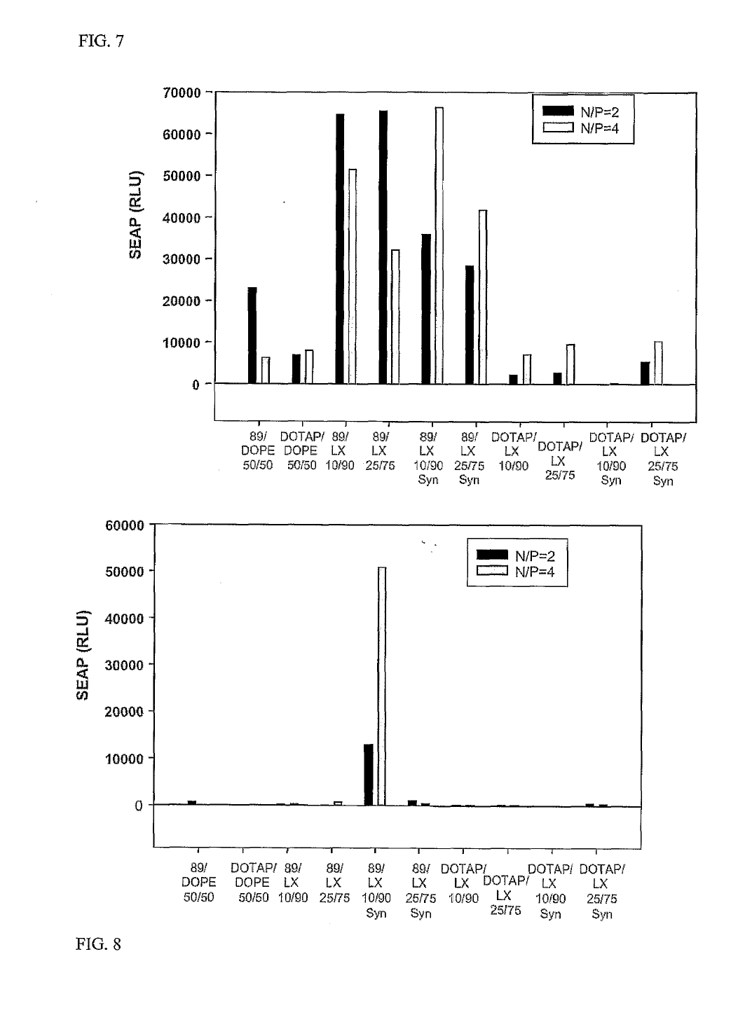

SDC (See U.S. Pat. No. 5,840,710, FIG. 7, No. 89) is prepared as follows. A solution of 3-benzyloxy-1,2-propanediol (1.00 g, 5.49 mmol) in THF (20 mL) was added to a suspension of sodium hydride (60% w/w in oil, 550 mg, 13.725 mmol) in THF (30 mL) and allowed to reflux overnight under dry nitrogen. A solution of dodecyl methane sulfonate (3.39 g, 12.078 mmol) in THF (20 mL) was added and the reaction was refluxed for another two days. After cooling to room temperature the reaction was filtered through a bed of Celite, rinsing with THF. The filtrate was reduced in vacuo to a yellow oil which was redissolved in diethyl ether (100 mL). The ether solution was washed with 0.1N NaOH (30 mL) and dH2O (2.times.30 mL). The organic layer was dried over magnesium sulfate, filtered and reduced in vacuo to a red-brown oil. The crude material was purified by flash column chromatography (300 g silica gel) eluting with 3% ethyl acetate/hexanes. The desired product was isolated as a pale yellow oil and characterized by ¹H NMR as 3-OBn-1,2-dilaurylglycerol (1.70 g, 60%). 3-OBn-1,2-dilaurylglycerol (1.70 g, 3.28 mmol) in ethanol (100 mL) was stirred with 10% Pd/C (250 mg, 15 wt %) under a hydrogen atmosphere for 24 hours. The reaction was flushed with nitrogen and filtered through Celite, rinsing with ethanol, to remove the catalyst. The filtrate was reduced in vacuo to a solid. The crude material was purified by flash column chromatography (140 g silica gel) eluting with 10% ethyl acetate/hexanes. The desired product was isolated as a white solid and characterized by ¹H NMR as 1,2-dilaurylglycerol (1.23 g, 88%).

A 1.93M solution of phosgene in toluene (0.77 mL, 1.49 mmol) was added to a solution of 1,2-dilaurylglycerol (580 mg, 1.35 mmol) and N,N-diisopropylethylamine (0.26 mL, 1.49 mmol) in methylene chloride (10 mL) and stirred overnight. A solution of N¹,N¹²-di-CBz-spermine.2HCl (734 mg, 1.35 mmol) in 60:25:4 chloroform/methanol/water (80 mL) was added. After 3 hours another equivalent of N,N-diisopropylethylamine (0.26 mL, 1.49 mmol) was added. An additional 0.5 equivalents of N,N-diisopropylethylamine (0.13 mL, 0.75 mmol) was added three hours later and the reaction was allowed to stir overnight under nitrogen at ambient temperature. The reaction was washed with 1M NaOH (20 mL) and dH2O (15 mL). The organic layer was separated, dried over magnesium sulfate, filtered and reduced in vacuo to a white solid. The crude material was purified by flash column chromatography (125 g silica gel) eluting with 90:10:0.5 chloroform/methanol/ammonium hydroxide. The desired product was isolated as an oil and characterized by ¹H NMR as 1-(N⁴-(N¹,N¹²-di-CBz-spermine))-2,3-dilaurylglycerol carbamate (188 mg, 15%).

The 1-(N⁴—(N¹,N¹²-di-CBz-spermine))-2,3-dilaurylglycerol carbamate (188 mg, 0.203 mmol) was dissolved in glacial acetic acid (10 mL) and stirred with 10% Pd/C (45 mg, 24 wt %) under a hydrogen atmosphere for 5 hours. The catalyst was removed by vacuum filtration rinsing with 10% acetic acid/ethyl acetate (10 mL) The filtrate was reduced to an oil by rotary evaporation. The resulting oil was dissolved in 10% methanol/chloroform (85 mL) and was washed with 1M NaOH (15 mL) and dH2O (10 mL). The organic layer was separated, dried over magnesium sulfate, filtered and reduced in vacuo to an oil. The product was characterized by ¹H NMR as 1-(N⁴-spermine)-2,3-dilaurylglycerol carbamate (125 mg, 94%).

Other amphiphiles of the invention may be prepared according to procedures that are within the knowledge of those skilled in art.

### EXAMPLE 2

### CLC/SDC Emulsion Protocol

1. SDC, CLC and α-tocopherol were combined in CHCl3/MeOH at 10:90 ratio.

2. Solvent is removed by evaporation.

3. Emulsion is hydrated in bicarbonate buffer pH 7.8 at a concentration of 10 uM lipid.

4. Emulsion is either used fresh or stored frozen at −20° C.

### EXAMPLE 3

### In Vitro Transfection Protocol

Cells were plated at 50% confluency in 24 well plates 18 hrs before transfection*
Plasmid DNA is isolated using a Qiagen kit. It is dialyzed against TE buffer.
1.65 ug of plasmid DNA is added to 55 or 110 nmoles of lipid in 0.33 ml.
After 10 minutes, complexes are diluted with 3 ml of serum free media.
1 ml of diluted transfection complexes are added per well.
After 1 hr incubation, transfection complexes are removed and media is replaced with complete media.
For human placental alkaline phosphatase assay, media is sampled 24, 48 and 72 hrs after addition of transfection complexes. For GFP expression, pictures were taken 24 hrs after transfection
   *Rat primary neurons were 8 day old cultures prior to gene transfer.

### EXAMPLE 4

### Cationic Amphiphile/CLC Formulation

cationic amphiphile according to Structure I or Structure II, or a combination thereof;

—CLC; and α-tocopherol; wherein said cationic amphiphile, CLC and α-tocopherol are present in a mole ratio of 5-15:85-95:0.5-2.0, respectively.

### EXAMPLE 5

### CLC Formulation

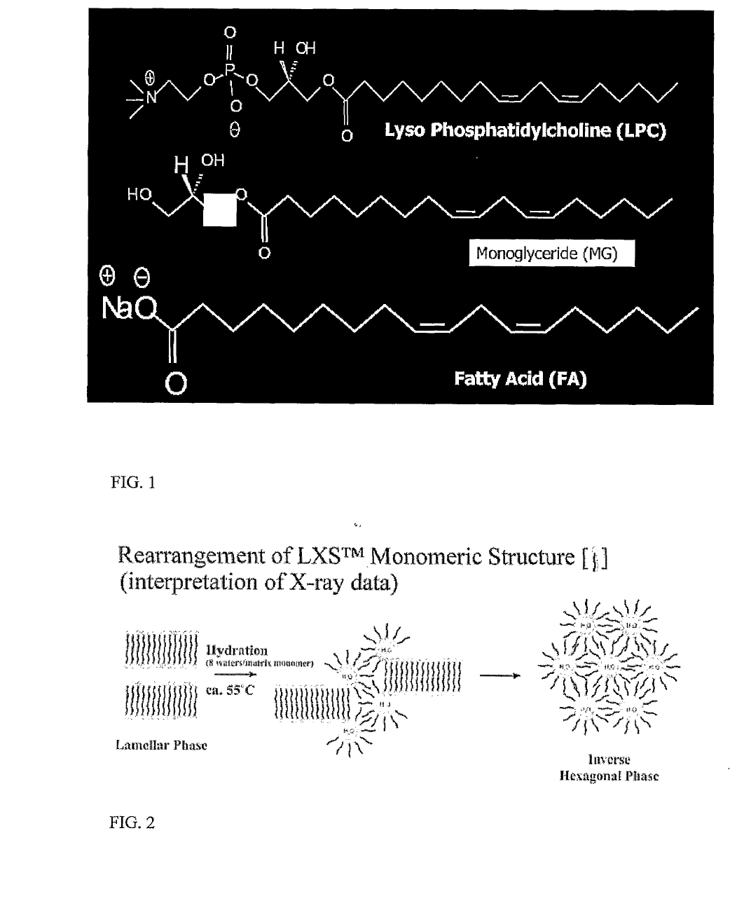

A particularly preferred CLC formulation that is combined with cationic amphiphiles is shown in FIG. 1 and whose tradename is LYM-X-SORB™ or LXS™. The mole ratios of Lyso Phosphatidylcholine (LPC), monoglyceride (MG) and fatty acid (FA) of LXS is 1:4:2, respectively. FIG. 2 shows the ability of LXS Monomeric Structure to rearrange into an inverse hexagonal phase. Some of the physical characteristics of LXS are as follows:
Eutectic organization demonstrates a single melting point which is lower than the melting points of the individual components.
LXS™ adopts lamellar structural organization at approximately zero water content.
LXS™ adopts purely hexagonal arrangement as water content approaches and exceeds 8 moles of water per mole of LXS™.
The LXS™ matrix can be a liquid or solid at room temperature by varying the unsaturation of the fatty acids.

### EXAMPLE 6

### Transfection into Cells

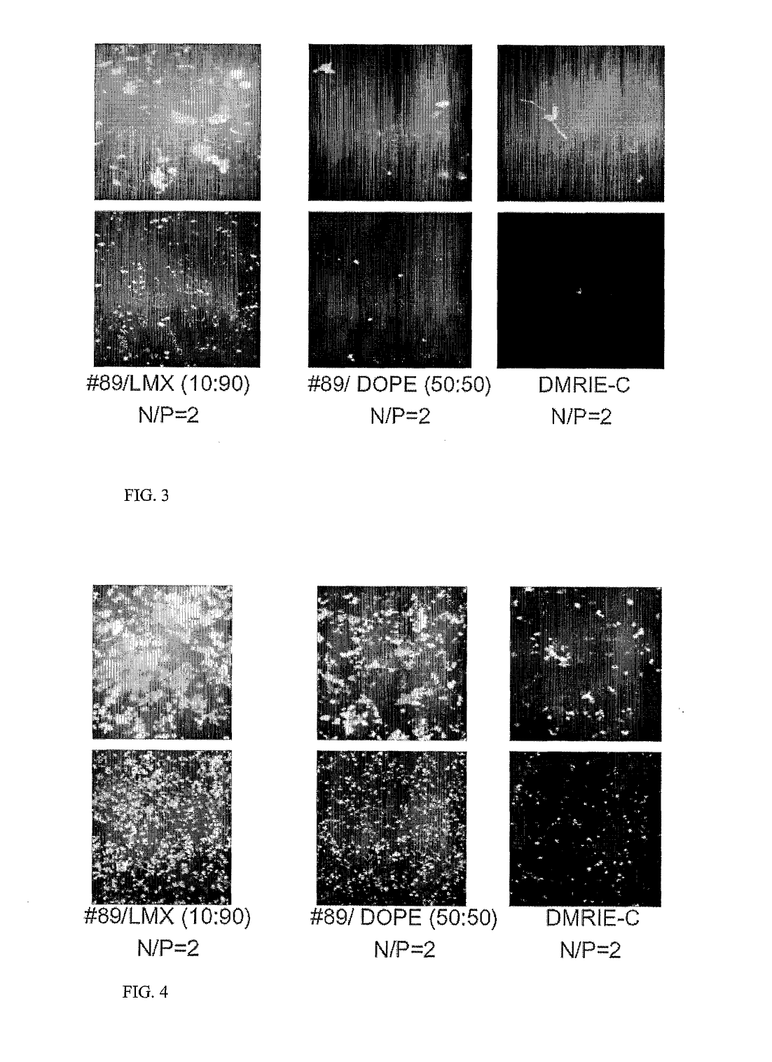

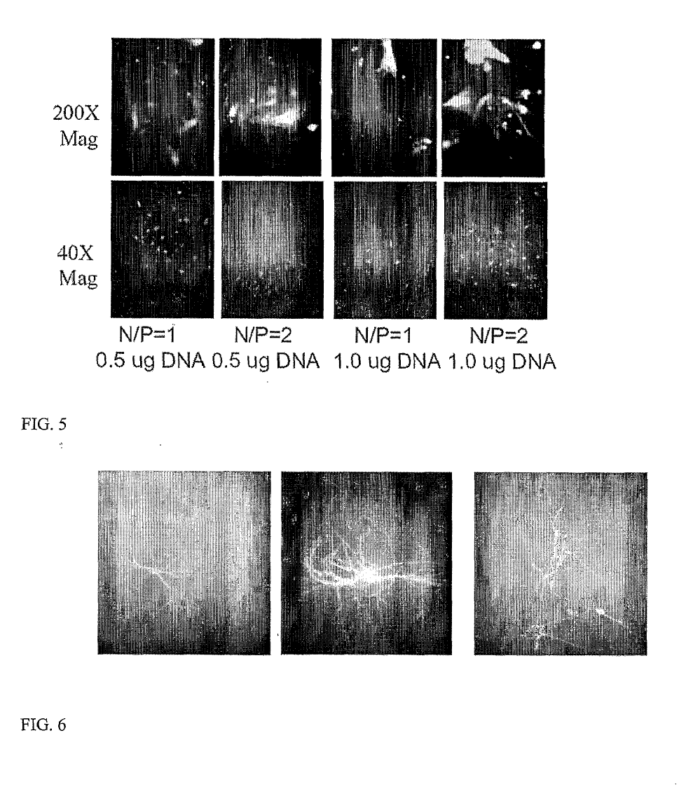

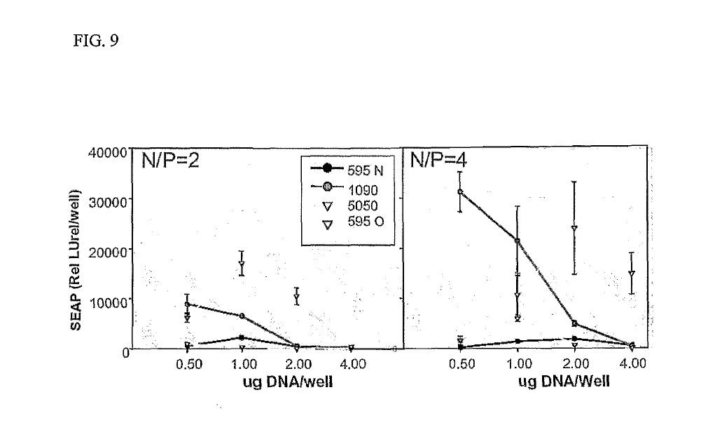

Utilizing the basic transfection protocol outlined in Example 3 above (note that such protocol is not intended to be limiting, and that other protocols may be used or developed by those skilled in the art in view of the teachings herein), different cells were treated with different formulations of 1-(N⁴-spermine)-2,3-dilaurylglycerol carbamate (125 mg, 94%) (designated as Lipid 89 or #89), LXS, and α-tocopherol complexed with a polynucleotide encoding an expressible GFP product. The results of these experiments are illustrated in FIGS. 3-9. FIG. 3 shows photographs of 3T3 cell transfection. FIG. 8 pertains to a graph of the results of 3T3 cell transfection. FIG. 9 shows effect of charge ratio on DNA dose response curve on 3T3 cells. FIG. 4 shows photographs of A293 cell transfection. FIG. 7 pertains to a graph of the results of A293 cell transfection. FIG. 5 shows transfection of human umbilical vascular endothelial cells. FIG. 6 shows transfection of rat neural cells.

### EXAMPLE 7

### Formation of Stable Transfection Complex

Plasmid DNA at 0.9 mg/ml in 40% ethanol, 5% dextrose is condensed with the spermine cationic amphipliles at a mole ratio of 2 cationic spermine amphiphiles to 1 DNA nucleotide. LXS suspended in 40% ethanol/α-tocopherol is added to the condensed plasmid DNA at a ration of 1 mole of cationic amphiphiles to 9 moles of LXS. At this point other molecules can be introduced into the suspension such a lipopeptide targeting ligand that upon incorporation into the transfection complexes will bind to a cell surface receptor and trigger site directed gene transfer to the cells expressing peptide binding receptor. The ethanol is removed by dialysis against PBS. (It can also be removed by lyophilization. This will require freezing the suspension to −50° C. and removing the ethanol under vacuum. Transfection complexes are then formed upon hydration of the lyophilized pellet). The dialyzed transfection complexes are ready to be used for transfection of cells or can be stored in the refrigerator as a suspension for up to 1 month.

### EXAMPLE 8

### Evaluation of the Lipid Formulation Using an Adult Mouse Eye Model Simulating Induction of Retinal Detachment Following Ablation Using Laser Surgery

Macular degeneration is a worsening of the retina near the optic nerve at the back of the eye. The retina is the area of the eye that sees detail at the center of the field of vision. There are two types of macular degeneration: wet and dry. The dry type is more common and cannot be treated. The wet type is caused by abnormal blood vessels that form under the retina and leak fluid or blood under and into the macular. If discovered early enough, the doctor can destroy these abnormal blood vessels by laser treatment. However, the same problem reoccurs a few months later due to the wound healing process. Also repeated surgeries results in irreparable scaring that leads to permanent blindness.

In the mouse eye model, the retina is lasered and hypervascularization is induced leading to retinal detachment. An expression plasmid encoding a ribozyme that cleaves the mRNA of IGF receptor was formulated with the CLC-SDA at a final plasmid DNA concentration of 0.2 mg/ml. The expression of the ribozyme is under the control of a proliferating endothelium promoter. This promoter restricts expression to only proliferating endothelium (promoter is covered in another patent). 0.0005 ul of formulation was injected into the lasered eye. Expression of a luciferase reporter gene used as a control was detected by immunohistochemistry out to 21 days after administration. Expression of the ribozyme resulted in an approximate 50% reduction in hypervascularization of the lasered eye compared to the non-plasmid treated control and a control treated only with the luciferase expressing plasmid. Accordingly, another aspect pertains to a method of treating or preventing macular degeneration comprising administering a lipoplex composition to an eye in need.

### EXAMPLE 9

### Peptide-Mediated Gene Transfer of Lipid/DNA-Based Complexes to Cells Expressing CXC-Receptor 4

Materials and Methods

Cell cultures and reagents. The Rat Glioma 2 (RG2) cell line (ATCC, Manassas, Va.) was maintained in Dulbecco's modified Eagle's medium (DMEM) with 4 mM L-glutamine adjusted to contain 1.5 g/L sodium bicarbonate and 4.5 g/L glucose, 10% fetal bovine serum and 10% PenStrep (Cellgro, Antioch, Ill.) at 37° C. and 5% CO₂. The Human Umbilical Vein Endothelial Cells (HUVECs) will be maintained in EGM-1 Bulletkit-medium composed of endothelial cell basal medium-1 (EBM-1 medium) supplemented with ascorbic acid, FBS, hydrocortisone, human epidermal growth factor (hEGF), gentainycin sulfate (GA-1000) and heparin as described by the manufacturer (Cambrex, Walkersville, Md.) at 37° C. and 5% CO₂. HUVECs will not be used after passage 10. The cells are cultured and maintained in a 75 cm² flask (Nunc, Rochester, N.Y.). One day before the experiment cells are washed with 1 ml of Trypsin-EDTA (Cellgro, Antioch, Ill.) and then removed from the flask with 2 ml of Trypsin-EDTA and plated in 6- or 24-well plates (Nunc, Rochester, N.Y.) at a density of 0.5×10⁶ or 0.05×10⁶ cells per well, respectively.

Flow Cytometry. Cells were grown in six-well plates in media with and without recombinant rat VEGF-164 (rrVEGF164, R&D systems Inc, Minneapolis, Minn.) and removed from the plate with a cell-dissociation solution (Mediatech, Herndon, Va.). After washing the cells with PBS containing 1% BSA and 0.1% sodium-azide they were incubated with 10 μg primary antibody (anti-rat CXCR4 polyclonal; Torrey Pines Biolabs, Houston, Tex.) in PBS with 1% BSA and 0.1% sodium-azide on ice for 30 minutes. Negative control antibodies consisted of appropriately matched isotypes at the same concentrations as primary antibodies. Cells were washed twice with cold PBS containing 1% BSA and 0.1% sodium-azide and incubated with 20 μg secondary antibodies conjugated to fluorescein-5-isothiocyanate (FITC) (Jackson Immunoresearch, West Grove, Pa.) on ice for 30 minutes. After two more washes cells were analyzed on the FACSort (BD Biosciences, San Jose, Calif.). Cells were gated via forward and side scatter dot plotting to exclude debris. The intensity of fluorescence was determined for 10,000 cells. Histograms recorded for gated cells were analyzed using WINMDI 2.8 software (J. Trotter; http://facs.scripps.edu). The percentage gated cells was compared to that in isotype antibody control.

Derivatization of peptide to phospholipid. 4-F-Benzoyl-TE140.11 (Tamamura, H., Hiramatsu, K., Mizumoto, M., Ueda, S., Kusano, S., Terakubo, S., Akamatsu, M., Yamamoto, N., Trent, J. O., Wang, Z., Peiper, S.C., Nakashima, H., Otaka, A. & Fujii, N. (2003) Org Biomol Chem 1, 3663-9) was kindly provided to us by Drs. Fujii and Tainamura. The targeting ligand was incorporated into lipid-based vesicles by coupling the peptide covalently to 1,2-Dioleoyl-sn-Glycero-3-Phosphoethanolamine-N-[4-(p-maleimidophenyl)butyramide] (DOPE-MPB). Briefly, the peptide was reacted with a 20-fold molar excess of N-Succinimidyl-5-acetylthioacetate (SATA) (Pierce, Rockford, Ill.) in 70% EtOH at pH=7.6. Derivatization was confirmed using the fluorescamine-assay for primary amines. Briefly, to peptide in solution 0.5 ml of fluorescamine in acetone (0.3 mg/ml) was added. After 10 minutes fluorescence was measured at λₑₓ=390 nm and λₑₘ=475 nm to confirm loss of the reactive amine (lysine 7). The modified peptide now contained a protected sulfhydryl group that was deprotected with a deprotection-solution containing an excess of hydroxylamine (0.5 M hydroxylamine, 0.5 M HEPES, 25 mM EDTA, pH=7.4). After deprotection the reactive thiol was reacted with equimolar amounts of DOPE-MPB to form a stable thio-ether. The reaction-progress was monitored by Thin-Layer-Chromatography (TLC). The normal phase plate was run in CHCl₃:MeOH:H₂O 65:25:4 and after reaction was complete the lipopeptide was purified using a double extraction as described by Folch (Folch, J., Ascoli, I., Lees, M., Meath, J. A. & Le, B. N. (1951) J Biol Chem 191, 833-41).

Cell associated fluorescence measurement. The lipopeptide was incorporated into liposomes at 20 mole percent. The other liposome-components consisted of 45 mole % 1,2-Dioleoyl-sn-Glycero-3-Phosphocholine (DOPC), 30 mole % cholesterol (CHOL) and 5 mole % 1,2-Dioleoyl-sn-Glycero-3-Phosphoethanolamine —N-(7-nitro-2-1,3-benzoxadiazol-4-yl) (DOPE-NBD) as a fluorescent marker (all lipids from Avanti Polar Lipids (Birmingham, Ala.)). After a one hour incubation of the liposomes with HUVE-cells; cells were thoroughly washed with PBS and cell-associated binding was measured by fluorescence after lysing the cells.

Reporter gene plasmid vectors. For experiments with plasmid DNA encoding for firefly luciferase, pLC0888 was used (Li, S., MacLaughlin, F. C., Fewell, J. G., Li, Y., Mehta, V., French, M. F., Nordstrom, J. L., Coleman, M., Belagali, N. S., Schwartz, R. J. & Smith, L. C. (1999) Gene Ther 6, 2005-11). For experiments with plasmid encoding for GPF, gWIZ-GFP vector (Gene Therapy Systems, San Diego, Calif.) was used. gWIZ-GFP vector was purified using the Qiafilter plasmid purification (Qiagen, Valencia, Calif.).

Preparation of lipoplexes. The lipid-based transfection-reagent (Genzyme lipid 89:Lym-X-Sorb:Vitamin E 10:89:1) was prepared aseptically by mixing 8 μl of Genzyme lipid 89 (25 mg/ml in chloroform or 0.038 μmoles/μl) (Genzyme, Cambridge, Mass.); 7.5 μl Lym-X-Sorb (138 mg/ml in chloroform or 0.365 μmoles/μl) (Avanti Polar Lipids, Birmingham, Ala.) and 2.2 μl Vitamin E (5.905 mg/ml or 0.0137 μmoles/μl) (Sigma, St Louis, Mo.) in a clean reagent-tube. Once the lipids were thoroughly mixed the solvent was evaporated using argon. Residual solvent was removed by placing the vial under a vacuum in the desiccator for 30 mins. The dried lipid-film was hydrated with 3 ml of HEPES Buffered Saline (HBS; 7.5 mM HEPES, 150 mM NaCl). After hydration the transfection-mixture was bath-sonicated for 5 minutes. For experiments with fluorescently tagged lipoplexes 0.1% Lym-X-Sorb was switched out for 1,2-Dioleoyl-sn-Glycero-3-Phosphoethanolamine-N-(Lissamine Rhodamine B Sulfonyl) (DOPE-Rhodamine). For experiments with targeted complexes 20% Lym-X-Sorb was switched out for purified lipopeptide prepared as described above.

Transfections. For the transfection, 3 μg of DNA was brought up to a volume of 180 μl with HBS and 180 μl transfection-reagent was added; this resulted in a lipid:DNA molar ratio of 2. The cells were washed with serum-free RG2-medium. After DNA/Lipid complexation was complete (˜15 minutes) 3 ml of serum-free medium was added to the DNA/Lipid mixture and 1 ml of diluted transfection-complex was added to each well ([DNA]=1 μg/well). After a 3 hour incubation serum-free medium was replaced with full media.

Luciferase-Assay. 48 hrs after transfection cells were removed from the incubator and washed with DPBS without Calcium and Magnesium (Cellgro, Antioch, Ill.). 150 μl of lysisbuffer (0.1 M potassium phosphate buffer; 1% Tx-100; 1 mM DTT; 2 mM EDTA; pH=7.8) (Sigma, St. Louis, Mo.) was added to each well and the plate was stored at −80° C. for 10 minutes or until ready to be assayed. The cell-lysate was thawed at room temperature and transferred to microfuge tubes. The cell-lysate is centrifuged at 13,200 rpm for 5 minutes. 40 μl of cell-lysate is added to each well of the assay-plate (Whatman, Clifton, N.J.). 100 μl substrate-buffer (30 mM Tricine; 3 mM ATP; 15 mM MgSO₄; 10 mM DTT; pH 7.8) and 100 μl of 1 mM D-luciferin pH=6.2 (Sigma, St. Louis, Mo.) was added to each well and the plate was run on a luminometer (Dynex Technologies, West Sussex, UK). A standard-curve of recombinant luciferase diluted in lysisbuffer was run simultaneously were 100 ng of recombinant luciferase corresponded to 0.5*10⁶ relative light units.

All results were normalized to total cell protein by measuring protein-concentration of all cell-lysates using the BCA-assay (Pierce, Rockford, Ill.).

Blood vessel organ culture. Animal-procedures were done under the approval of the local committee for animal experiments. Sprague Dawley rats were euthanized by inhalation of Isoflurane overdose. The mesentery was dissected and placed in ice-cold DBPS-buffer. Mesenteric artery segments of about 10 min length were dissected and mounted in an organ culture system.

CXCR4-expression on cells in the blood vessel organ culture was determined by means of indirect immunohistochemistry. Briefly, after incubating the arteries with medium supplemented with 100 ng/ml rrVEGF164 overnight, 10 μg rabbit anti-rat CXCR4 polyclonal antibody was perfused through the artery in a volume of 1 ml DPBS and incubated for 30 minutes. After the luminal side of the artery was washed by perfusing 1 ml cold DPBS through the arteries, 20 μg Goat anti-rabbit secondary antibody conjugated to fluorescein-5-isothiocyanate (FITC) (Jackson Immunoresearch, West Grove, Pa.) was perfused as well and incubated for 30 minutes. After washing the arteries fluorescent micrographs were taken.

For the experiments with the fluorescently labeled transfection-complexes empty plasmid-DNA was complexed with the transfection reagent and diluted up to 1 ml with medium. The 1 ml was perfused slowly through the arteries and washed with 3 ml of DPBS after a 30 minute incubation. After washing fluorescent micrographs were taken. For the vessel-transfection experiments lipoplexes expressing GFP were prepared, 1 μg of DNA was perfused in a volume of 1 ml and after 3 hours the arteries were washed with DPBS. Reporter-gene expression was assessed after 48 hours.

### Results

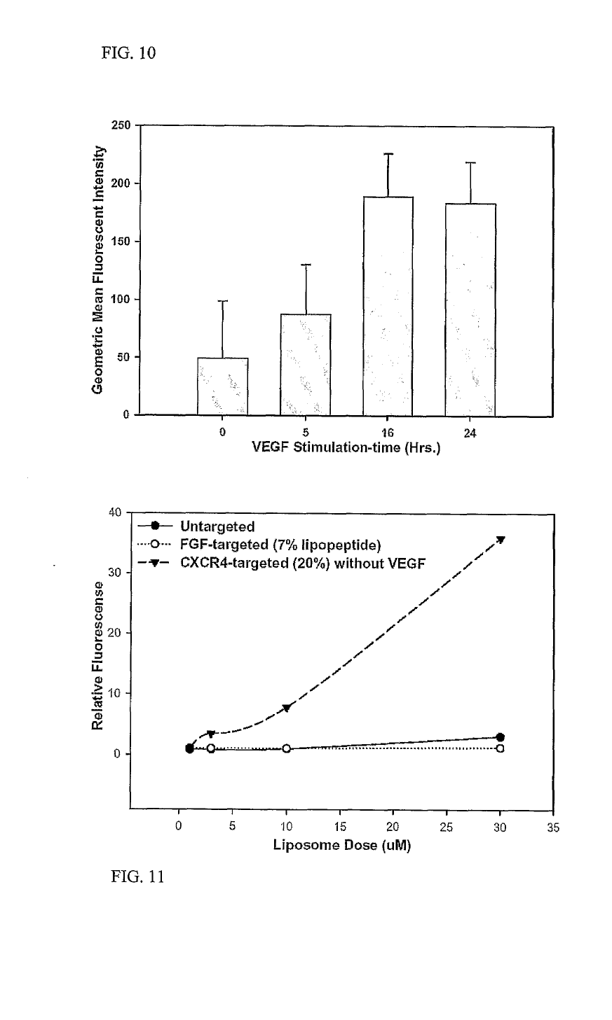

Incubation with recombinant rat VEGF164 upregulates CXCR4 on HUVECs resulting in receptor-specific binding of CXCR4-targeted liposomes in vitro. In order to successfully enhance specificity of lipid-mediated gene-delivery the receptor being targeted should be abundantly expressed on the cell-surface of the target-tissue. The inventor's flow cytometry results showed that HUVECs being cultured in medium with no VEGF present showed only a low surface-expression of CXC-receptor 4. However, when rrVEGF164 was supplemented into the medium, surface-expression of the chemokine-receptor gradually increased until it reached a plateau after about 16 hours. The plateau-level of CXCR4-expression reflected a five-fold increase in the number of receptors expressed on the surface of HUVECs compared to non-VEGF stimulated controls (FIG. 10). This increase of CXCR4-expression on HUVEC in response to VEGF has been reported previously and has successfully been validated in our in vitro model-system for endothelial cells. This high surface-expression of receptor in VEGF-stimulated endothelial cells in combination with a low surface-density in the normal state of endothelial cells is an excellent tool to show function of receptor-density for cell-specific uptake. The inventor's liposome-binding studies in HUVECs showed that non-VEGF treated endothelial cells bound CXCR4-targeted liposomes to the same extend as untargeted control-liposomes. However, when the cells were incubated overnight with 100 ng/ml rrVEGF164, the CXCR4-targeted liposomes bind increasingly to the surface of HUVECs in a dose-dependent matter as determined by measuring the total cell-associated fluorescence. At the highest dose of 30 μM lipid, CXCR4-targeted liposomes bound nine-fold more effectively then their untargeted controls (FIG. 11).

It is clear from these data that the number of receptors for our targeting ligand on HUVECs was increased five-fold by an overnight incubation of the cells with rrVEGF164. This increase of receptor-number in its turn, lead to specific binding of targeted liposomes to the endothelial cells. Specificity was clearly shown by the fact that the same targeted liposomes did not bind to cells expressing lower numbers of surface-receptors (i.e. not incubated with rrVEGF164).

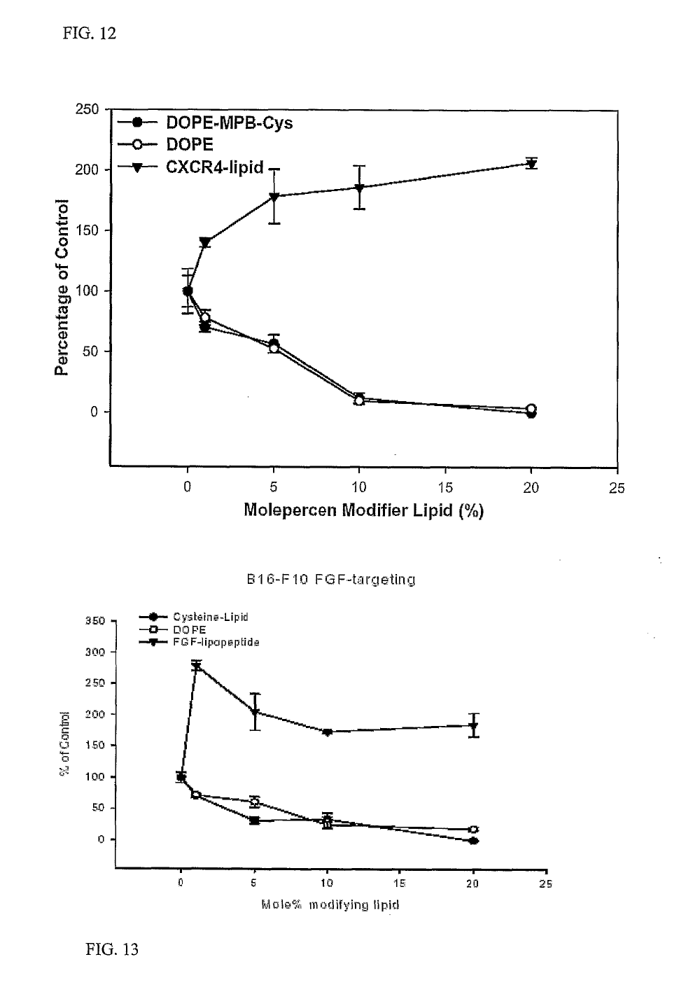

Transfection With CXCR4-ligand targeted lipoplexes leads to increased transfection-efficiency in vitro. Whether increased specific binding to cells expressing the targeted receptor leads to increased transfection-efficiency in vitro is tested by transfecting cells with targeted and untargeted lipoplexes expressing the reporter-enzyme luciferase. Many tumor cell-lines express CXCR4, including the RG2 cell-line (as confirmed by flow cytometry; data not shown). These cells are efficiently transfected with lipoplexes prepared as described in the materials and methods section. Transfection of cells with untargeted lipoplexes coding for GFP at a dose of 1 μg DNA per 50,000 cells typically leads to a 30-40% transfection-efficiency after 48 hours (data not shown). However, whenever the lipid-composition of the optimized formulation was changed by titrating in cysteine-modified DOPE-MPB (DOPE-MPB-Cys) or DOPE instead of Lym-X-Sorb (i.e. modification of helper-lipids); the transfection-efficiency decreased steadily. Even at low concentrations of DOPE-MPB-Cys and DOPE (1 molepercent) transfection-efficiency as measured by luciferase-expression after 48 hours (1 μg per well) was decreased by about 30% (100% being 4*10⁴ RLU equivalent to 35 ng recombinant luciferase per well). At higher concentrations of modification-lipid (20 molepercent) transfection-efficiency even decreased to background levels (DOPE-MPB-Cys: 0.007±0.004; DOPE: 3.9±1.1% of control)(FIG. 12). The opposite effect was observed when DOPE-MPB modified with the CXCR4-targeting ligand (DOPE-CXCR4) was titrated into the formulation. Even at 1 molepercent the luciferase-expression was increased by 40%; at the higher mole-percentages the expression was about two-fold increased compared to unmodified control (FIG. 12). A similar result was observed for the FGF receptor binding lipopeptide under the same experimental conditions but using B16 melanoma cells. These cells were used because they have a higher FGF receptor density than the RG2 cells. The results are shown in FIG. 13. The same trend in increased transfection efficiency as a function of the mol % of bFGF receptor binding lipopeptide was observed as that of the CXCR 4 binding lipopeptide. Addition of either DOPE or cysteine-DOPE decreased luciferase expression in these cells as well. Accordingly, another aspect of the invention is directed to lipoplexes comprising one, two, three or more cell targeting molecules, such as, but not limited to lipopeptides designed to target a cell receptor. The addition of such cell targeting molecules will increase the transfection efficiency as well as the specificity of the lipoplexes.

These data show that a transfection-complex which is rendered non-active after introduction of modifier-lipids is restored to its original activity, improved even, after introducing a modifier lipid conjugated to a high affinity ligand specifically recognizing a receptor highly expressed on the cell-surface of the target-cell.

Incubation with recombinant rat VEGF164 upregulates CXCR4 on mesenteric endothelial cells of the blood vessel organ culture. To test the interaction of targeted lipoplexes with endothelial cells in a more biologically relevant context before testing them in vivo our laboratory has developed a organ culture of mesenteric rat-arteries. The dissected arteries from the mesentery can be mounted on a custom-made perfusion system and kept viable for up to four days. This provides a three-dimensional model-system which can be monitored continuously using standard microscopy far more relevant then 2-dimensional cell cultures. To validate if the CXC-receptor 4 was regulated by VEGF in the same way as observed in cell cultures; arteries were incubated with 100 ng/ml rrVEGF164 overnight and stained for CXCR4 using indirect immunohistochemistry. Also in the organ cultures of mesenteric arteries it could be clearly seen that CXCR4 is only abundantly expressed on blood vessel endothelial cells after incubation with VEGF as indicated by the green fluorescence. The arteries which were not incubated with VEGF or negative controls only showed a faint green auto-fluorescence, clearly distinguishable from CXCR4-positive staining.

This experiment shows that rat mesenteric arteries can be isolated and kept viable for up to four days. Moreover, CXCR4-expression is controlled in a similar fashion as observed in HUVECs; giving us the opportunity to study the interaction of our different formulations, especially the targeted lipoplex, in a biologically very relevant three-dimensional model-system.

CXCR4-targeted lipoplexes bind specifically to endothelial cells expressing the receptor in the blood vessel organ culture. To assess if CXCR4-ligand targeted lipoplexes would bind specifically to endothelial cells of mesenteric arteries in the organ culture we formulated rhodamine-labeled transfection-complexes with empty vector-plasmid (pVC 1157). After slowly perfusing the labeled complexes incubating for 30 minutes and washing the artery-lumen with three volumes of DPBS red fluorescence was visualized. Again arteries not incubated with rrVEGF164 overnight and arteries incubated with untargeted complexes only displayed very a very faint background fluorescence in stark contrast to the targeted complexes. Endothelial cell labeling was clearly visible as red fluorescence along the length of the artery.

Even with normal fluorescent microscopy it was possible to take fluorescent micrographs of different sections of the artery along the z-axis (i.e. top, middle and bottom section; pictures from top and bottom sections not shown), clearly demonstrating the three-dimensional nature of the model.

GFP-expression in mesenteric arteries only with CXCR4-targeted lipoplexes after stimulation with rrVEGF164. Because increased binding of the transfection complex to the target-cells doesn't necessarily translate into more efficient transfection we have transfected mesenteric endothelium with GFP. In the vessel not treated with rrVEGF164 overnight there almost no cells expressing the fluorescent protein; in the VEGF-treated vessel however the targeted lipoplex was very successful in transfecting the endothelial cell-layer; indicated by the green fluorescence along the vessel-wall. Clearly, transfection is limited to the endothelial cells, smooth muscle cells remain unaffected. The results presented above clearly show that the CXC-receptor 4 is expressed on endothelial cells in vitro and in the blood vessel organ culture, especially in environments with high VEGF-concentrations. That makes the receptor an interesting target to use for site-specific gene-delivery. The developed targeted lipoplex can be used for the delivery of therapeutic genes to tumor vasculature, various solid tumors and other tissues expressing the receptor in high quantities. Furthermore the mesenteric artery organ culture has shown to be a biologically relevant model in which we have validated our in vitro receptor-expression, liposome-binding and transfection-results.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference in their entirety to the extent they are not inconsistent with the teachings herein.

1. A composition for facilitating delivery of a biological agent into a cell, said composition comprising:
a non-esterified fatty acid having 14-18 carbon atoms;
a monoglyceride which is a monoester of glycerol and a fatty acid having 14-18 carbon atoms;
a lysophosphatidyl choline in which the fatty acid moiety has 14-18 carbon atoms; and
a cationic amphiphile according to Structure I or Structure II.

2. The composition of claim 1, wherein said composition is an admixture of a first composition comprising a cationic amphiphile according to Structure I or Structure II, or combination thereof; and a second composition comprising a non-esterified fatty acid having 14-18 carbon atoms; a monoglyceride which is a monoester of glycerol and a fatty acid having 14-18 carbon atoms; and a lysophosphatidyl choline in which the fatty acid moiety has 14-18 carbon atoms; wherein said first and second compositions are present in a mole ratio of 5-15:85-95, respectively.

3. The composition of claim 2, wherein the second composition comprises a non-esterified fatty acid having 14-18 carbon atoms; a monoglyceride which is a monoester of glycerol and a fatty acid having 14-18 carbon atoms; and a lysophosphatidyl choline in which the fatty acid moiety has 14-18 carbon atoms, wherein the mole ratio of said monoglyceride to fatty acid is from about 2:1 to about 1:2 and the mole ratio of lysophosphatidyl choline to the sum of monoglycerides and fatty acid is from about 1:3 to about 1:12.

4. The composition of claim 1, wherein said composition further comprises α-tocopherol.

5. The composition of claim 2, wherein said composition is an admixture of a first composition comprising a cationic amphiphile according to Structure I or Structure II, or a combination thereof; a second composition comprising a non-esterified fatty acid having 14-18 carbon atoms; a monoglyceride which is a monoester of glycerol and a fatty acid having 14-18 carbon atoms; and a lysophosphatidyl choline in which the fatty acid moiety has 14-18 carbon atoms; and a third composition comprising α-tocopherol; wherein said first, second and third compositions are present in a mole ratio of 5-15:85-95:0.5-2.0, respectively.

6. The composition of claim 1, wherein the structure represented by R³—R¹—N—R²—R⁴ in Structure I is spermine or spermidine.

7. 7. The composition of claim 1 wherein said cationic amphiphile is 1-(N⁴-spermine)-2,3-dilaurylglycerol carbamate; 1-(N⁴-spermidine)-2,3-dilaurylglycerol carbamate; 1-(N⁴-spermine)-2,3-distearylglycerol carbamate; and 1-(N⁴-spermidine)-2,3-distearylglycerol carbamate.

8. The composition of claim 1, wherein the structure represented by R³—R¹—N—R²—R⁴ in Structure II is spermine or spermidine.

9. The composition of claim 8, wherein said cationic amphiphile is 1-(N⁴-Spermidine)-2,3-dilauroylglycerol carbamate; and 1-(N⁴-Spermidine)-2,3-distearoylglycerol carbamate.

10. A biologically active agent/cationic amphiphile complex, wherein said complex comprises
a non-esterified fatty acid having 14-18 carbon atoms;
a monoglyceride which is a monoester of glycerol and a fatty acid having 14-18 carbon atoms;
a lysophosphatidyl choline in which the fatty acid moiety has 14-18 carbon atoms; and
a cationic amphiphile according to Structure I or Structure II; and
a biologically active agent that comprises an anionic charge; wherein said biologically active agent and said cationic amphiphile comprise a noncovalent interaction.

11. The composition of claim 10, wherein said biologically active agent is a polynucleotide.

12. The composition of claim 11, wherein the polynucleotide is a recombinant DNA molecule.

13. The composition of claim 10, wherein said DNA consists of a gene.

14. A process for obtaining an expression product in vivo, comprising:
a) forming a complex with a non-esterified fatty acid having 14-18 carbon atoms; a monoglyceride which is a monoester of glycerol and a fatty acid having 14-18 carbon atoms; a lysophosphatidyl choline in which the fatty acid moiety has 14-18 carbon atoms; a cationic amphiphile according to Structure I or Structure II; and a polynucleotide;
b) delivering said complex to a cell in a mammal; and
c) expressing the polynucleotide.

15. The process of claim 14, wherein said delivering comprises intraocular injection of said complex, wherein said complex transfects into retinal cells.

16. The process of claim 14, wherein said cationic amphiphile is 1-(N⁴-spermine)-2,3-dilaurylglycerol carbamate; 1-(N⁴-spermidine)-2,3-dilaurylglycerol carbamate; 1-(N⁴-spermine)-2,3-distearylglycerol carbamate; and 1-(N⁴-spermidine)-2,3-distearylglycerol carbamate.

17. A process for delivering a biological agent in vivo, comprising:
a) forming a complex with a non-esterified fatty acid having 14-18 carbon atoms; a monoglyceride which is a monoester of glycerol and a fatty acid having 14-18 carbon atoms; a lysophosphatidyl choline in which the fatty acid moiety has 14-18 carbon atoms; a cationic amphiphile according to Structure I or Structure II; and a biological agent; and
b) delivering said complex to a cell in a mammal;

18. The process of claim 17, wherein said delivering comprises intraocular injection of said complex, wherein said complex transfects into retinal cells.

19. The process of claim 18, wherein said biological agent is a ribozyme.

20. The process of claim 17, wherein said complex further comprises a cell targeting lipopeptide.

21. The process of claim 20, wherein said complex further comprises two or more cell targeting lipopeptides.
